(19) United States
(12) Patent Application Publication
(10) Pub. No.: US 2024/0354712 A1
(43) Pub. Date: Oct. 24, 2024

(54) System and Method for managing patient appointments

(71) Applicant: Veeva Systems Inc., Pleasanton, CA (US)

(72) Inventors: Bonnie Bates, Kansas City, MO (US); Paul Hartwell, Olathe, KS (US)

(73) Assignee: Veeva Systems Inc., Pleasanton, CA (US)

(21) Appl. No.: 18/642,815

(22) Filed: Apr. 22, 2024

Related U.S. Application Data
(60) Provisional application No. 63/461,238, filed on Apr. 21, 2023.

(51) Int. Cl. G06Q 10/1093; G16H 40/20

(52) U.S. Cl. CPC G06Q 10/1095; G16H 40/20

(57) ABSTRACT

Systems and methods for managing patient appointments. A calendar alias may be created and added to the record of a first patient. The clinical trial site user may add the calendar alias of the first patient to a meeting request as an invitee, similar to adding an email address to the meeting request. The clinical trial site user may add other information to the meeting request, such as subject (visit, dose, consent, blood draw, etc.), time, location (office address, Zoom meeting link, etc.) and other invitees.

### CROSS-REFERENCE TO RELATED APPLICATION

This application relates and claims priority to provisional patent application No. 63/461,238, filed on Apr. 21, 2023, entitled System and Method for Managing Patient Appointments, which is hereby incorporated by reference herein for all purposes.

### BACKGROUND

The subject technology relates generally to managing patient appointments during one or more clinical trials.

Clinical trials are research studies on participants designed to answer specific questions about biomedical or behavioral interventions, including new treatments (such as drugs and medical devices). In addition to participants/patients, a clinical trial may involve a sponsor, which may be a governmental organization, or a pharmaceutical, medical device, or biotechnology company, and one or more clinical sites. Participants may come to a clinical site a number of times during a clinical trial for tests and treatments. It is desirable to improve efficiency of the process for managing patient appointments.

### SUMMARY

A method for managing patient appointments in a system comprising a patient information management system which maintains patient information. The method comprising: creating a first calendar alias for a first patient and adding the first calendar alias to information of the first patient in the patient information management system; displaying a first user interface for an appointment scheduling system in response to a request from a user; adding the first calendar alias of the first patient to a first meeting request as a first invitee; receiving the first meeting request at the patient information management system; routing the first meeting request the first patient by the patient information management system, based on the first calendar alias; and adding the first meeting request to the first patient's calendar.

### BRIEF DESCRIPTION OF THE DRAWINGS

FIG. 1 illustrates an example high level block diagram of a system for managing patient appointments during a clinical trial according to one embodiment of the present invention.

FIG. 2 illustrates an example block diagram of a computing device.

FIG. 3 illustrates an example high level block diagram of the clinical trial management server according to one embodiment of the present invention.

FIG. 4 illustrates an example high level block diagram of the patient information management server according to one embodiment of the present invention.

FIG. 5 illustrates an example high level block diagram of a user computing device.

FIG. 6 illustrates an example flowchart of a method for managing patient appointments during a clinical trial according to one embodiment of the present invention.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced without these specific details. In some instances, well-known structures and components are shown in block diagram form in order to avoid obscuring the concepts of the subject technology.

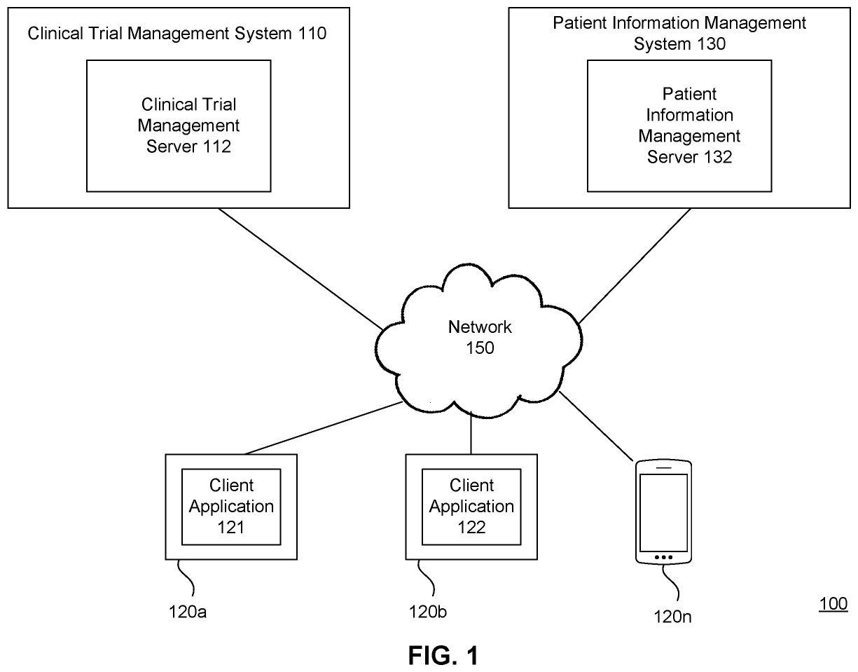

FIG. 1 illustrates an example high level block diagram of a system for managing patient appointments according to one embodiment of the present invention. As shown, the system 100 may include a clinical trial management system 110, a plurality of user computing devices 120a, 120b, . . . 120n, and a patient information management system 130 coupled to each other via a network 150. The clinical trial management system 110 may include a clinical trial management server 112. The patient information management system 130 may include a patient information management server 132. The network 150 may include one or more types of communication networks, e.g., a local area network (“LAN”), a wide area network (“WAN”), an intra-network, an inter-network (e.g., the Internet), a telecommunication network, and peer-to-peer networks (e.g., ad hoc peer-to-peer networks), which may be wired or wireless.

The user computing devices 120a-120n may be any machine or system that is used by a user to access the clinical trial management system 110 and the patient information management system 130 via the network 150, and may be any commercially available computing devices including laptop computers, desktop computers, mobile phones, smart phones, tablet computers, netbooks, and personal digital assistants (PDAs). A client application 121 may run from a user computing device, e.g., 120a, and access content in the content management system 110 via the network 150. A client application 122 may run from a user computing device, e.g., 120b, and access content in the patient information management system 130 via the network 150.

The clinical trial management server 112 is typically a remote computer system accessible over a remote or local network, such as the network 150. The clinical trial management server 112 could be any commercially available computing devices. A client application (e.g., 121) process may be active on one or more user computing devices 120a-120n. The corresponding server process may be active on the clinical trial management server 112. The client application process and the corresponding server process may communicate with each other over the network 150, thus providing distributed functionality and allowing multiple client applications to take advantage of the information-gathering capabilities of the content management system 110.

The patient information management server 132 is typically a remote computer system accessible over a remote or local network, such as the network 150. The patient information management server 132 could be any commercially available computing devices. A client application (e.g., 122) process may be active on one or more user computing devices 120a-120n. The corresponding server process may be active on the patient information management server 132. The client application process and the corresponding server process may communicate with each other over the network 150, thus providing distributed functionality and allowing multiple client applications to take advantage of the information-gathering capabilities of the content management system 110.

Although the clinical trial management server 112 and the patient information management server 132 are shown as two separate servers, it should be appreciated that they could be combined as one server.

In one implementation, the clinical trial management system 110 may be a multi-tenant system where various elements of hardware and software may be shared by one or more customers. For instance, a server may simultaneously process requests from a plurality of customers. In a multi-tenant system, a user is typically associated with a particular customer. In one example, a user could be an employee of one of a number of pharmaceutical companies which are tenants, or customers, of the clinical trial management system 110.

In one embodiment, the clinical trial management system 110 may run on a cloud computing platform. Users can access content on the cloud independently by using a virtual machine image, or purchasing access to a service maintained by a cloud database provider.

In one embodiment, the clinical trial management system 110 may be provided as Software as a Service (“SaaS”) to allow users to access the clinical trial management system 110 with a thin client.

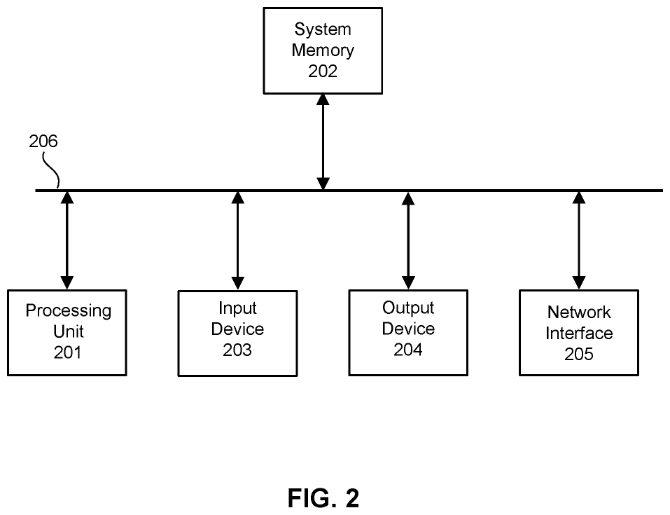

FIG. 2 illustrates an example block diagram of a computing device 200 which can be used as the user computing devices 120a-120n, the clinical trial management server 112 and the patient information management server 132 in FIG. 1. The computing device 200 is only one example of a suitable computing environment and is not intended to suggest any limitation as to scope of use or functionality. The computing device 200 may include a processing unit 201, a system memory 202, an input device 203, an output device 204, a network interface 205 and a system bus 206 that couples these components to each other.

The processing unit 201 may be configured to execute computer instructions that are stored in a computer-readable medium, for example, the system memory 202. The processing unit 201 may be a central processing unit (CPU).

The system memory 202 typically includes a variety of computer readable media which may be any available media accessible by the processing unit 201. For instance, the system memory 202 may include computer storage media in the form of volatile and/or nonvolatile memory such as read only memory (ROM) and/or random access memory (RAM). By way of example, but not limitation, the system memory 202 may store instructions and data, e.g., an operating system, program modules, various application programs, and program data.

A user can enter commands and information to the computing device 200 through the input device 203. The input device 203 may be, e.g., a keyboard, a touchscreen input device, a touch pad, a mouse, a microphone, and/or a pen.

The computing device 200 may provide its output via the output device 204 which may be, e.g., a monitor or other type of display device, a speaker, or a printer.

The computing device 200, through the network interface 205, may operate in a networked or distributed environment using logical connections to one or more other computing devices, which may be a personal computer, a server, a router, a network PC, a peer device, a smart phone, or any other media consumption or transmission device, and may include any or all of the elements described above. The logical connections may include a network (e.g., the network 150) and/or buses. The network interface 205 may be configured to allow the computing device 200 to transmit and receive data in a network, for example, the network 150. The network interface 205 may include one or more network interface cards (NICs).

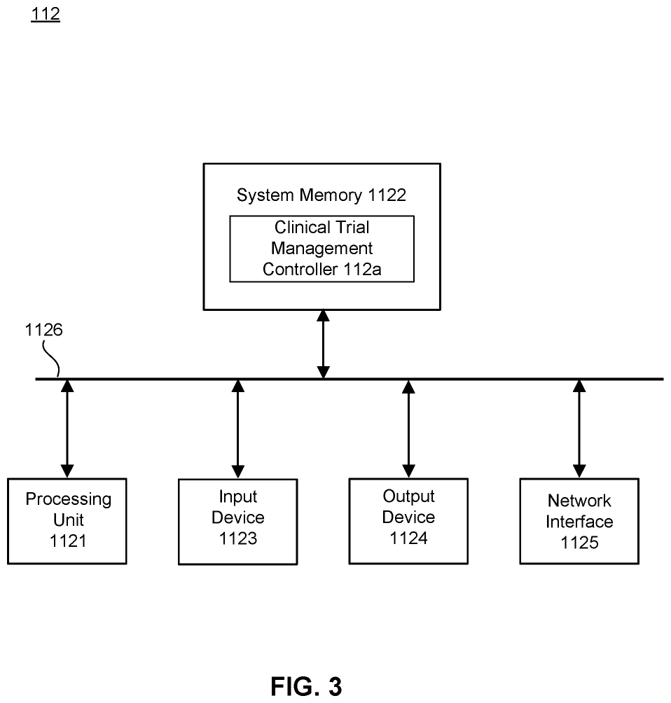

FIG. 3 illustrates an example high level block diagram of the clinical trial management server 112 according to one embodiment of the present invention. The clinical trial management server 112 may be implemented by the computing device 200, and may have a processing unit 1121, a system memory 1122, an input device 1123, an output device 1124, and a network interface 1125, coupled to each other via a system bus 1126. The system memory 1122 may store a clinical trial management controller 112a and a medical data collection controller 112b.

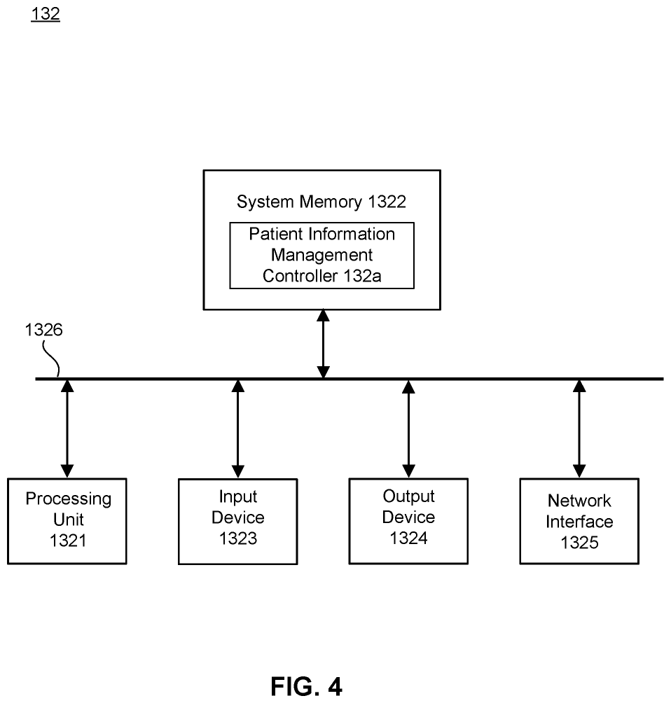

FIG. 4 illustrates an example high level block diagram of the patient information management server 132 according to one embodiment of the present invention. The patient information management server 132 may be implemented by the computing device 200, and may have a processing unit 1321, a system memory 1322, an input device 1323, an output device 1324, and a network interface 1325, coupled to each other via a system bus 1326. The system memory 1322 may store a patient information management controller 132a.

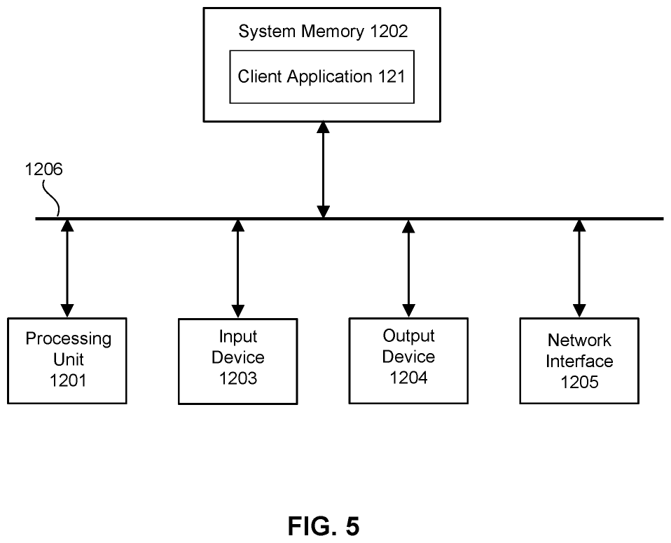

FIG. 5 illustrates an example high level block diagram of a user computing device (e.g., 120a) wherein the present invention may be implemented. The user computing device 120a may be implemented by the computing device 200 described above, and may have a processing unit 1201, a system memory 1202, an input device 1203, an output device 1204, and a network interface 1205, coupled to each other via a system bus 1206. The system memory 1202 may store the client application 121.

The client application 121 may be an application installed on a computing device, or a web application. Users at a clinical trial site may enter patient clinical trial information via the client application 121.

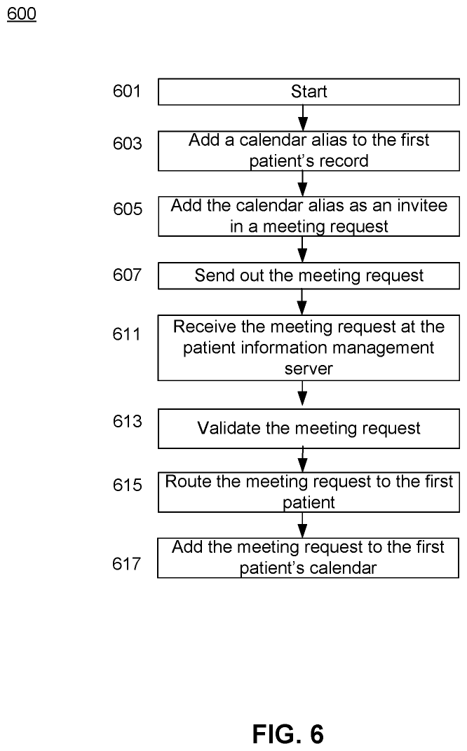

FIG. 6 illustrates a flowchart of a method for managing patient appointments according to one embodiment of the present invention. The process may be controlled by the clinical trial management server 112 and the patient information management server 132, and start at 201.

The clinical trial management system 110 may maintain clinical trial participants/patient information, which may include the patient's demographic information (e.g., name, age and sex), contact information (e.g., address, phone number and email address), health information and medical information. At 603, a calendar alias may be created and added to the record of a first patient. In one embodiment, the calendar alias may be formatted like an email address, including a unique identifier of the first patient, and the @ symbol. In one embodiment, the calendar alias may be associated with the patient's email address.

A user at a clinical trial site may want to schedule an appointment with the first patient. The clinical trial site user may be using the user computing device 120a. At 605, the clinical trial site user may login into an appointment scheduling system, and open an appointment scheduling user interface. The clinical trial site user may add the calendar alias of the first patient to a meeting request as an invitee, similar to adding an email address to the meeting request. The clinical trial site user may add other information to the meeting request, such as subject (visit, dose, consent, blood draw, etc.), time, location (office address, Zoom meeting link, etc.) and other invitees.

At 607, the clinical trial site user may send out the meeting request.

At 611, the meeting request may be received at the patient information management server 132.

At 613, the meeting request may be validated at the patient information management server 132 to make sure that it is from a valid user account associated with the clinical trial management system 110, instead of spam.

At 615, the meeting request may be routed to the first patient, by the patient information management server 132, based on the calendar alias.

At 617, the meeting request may be received at the application 122 on the first patient's user computing device 120b, which may add the meeting request to the first patient's calendar in the application 122, and alert the first patient about the meeting request. The application 122 may also alert the first patient about the appointment shortly before that.

The present invention allows the clinical trial site user to schedule the appointment with the first patient securely, without risking the first patient's personal information and medical information. The appointment can be displayed in the first patient's calendar in the application 122. The patient will get notifications about the upcoming appointment. If the appointment is a virtual appointment, the patient can join it conveniently by clicking on the meeting link in the meeting request.

The above-described features and applications can be implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

In this specification, the term “software” is meant to include firmware residing in read-only memory or applications stored in magnetic storage, which can be read into memory for processing by a processor. Also, in some implementations, multiple software technologies can be implemented as sub-parts of a larger program while remaining distinct software technologies. In some implementations, multiple software technologies can also be implemented as separate programs. Finally, any combination of separate programs that together implement a software technology described here is within the scope of the subject technology. In some implementations, the software programs, when installed to operate on one or more electronic systems, define one or more specific machine implementations that execute and perform the operations of the software programs. Examples of computer programs or computer code include machine code, for example is produced by a compiler, and files including higher-level code that are executed by a computer, an electronic component, or a microprocessor using an interpreter.

A computer program (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

It is understood that any specific order or hierarchy of steps in the processes disclosed is an illustration of example approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components illustrated above should not be understood as requiring such separation, and it should be understood that the described program components and systems can generally be integrated together in a single software product or packaged into multiple software products.

Various modifications to these aspects will be readily apparent, and the generic principles defined herein may be applied to other aspects. Thus, the claims are not intended to be limited to the aspects shown herein, but is to be accorded the full scope consistent with the language claims, where reference to an element in the singular is not intended to mean “one and only one” unless specifically so stated, but rather “one or more.” Unless specifically stated otherwise, the term “some” refers to one or more.

What is claimed is:

1. A computer-implemented method for managing patient appointments in a system comprising a patient information management system which maintains patient information, the method comprising:
creating a first calendar alias for a first patient and adding the first calendar alias to information of the first patient in the patient information management system;
displaying a first user interface for an appointment scheduling system in response to a request from a user;
adding the first calendar alias of the first patient to a first meeting request as a first invitee;
receiving the first meeting request at the patient information management system;
routing the first meeting request the first patient by the patient information management system, based on the first calendar alias; and
adding the first meeting request to the first patient's calendar.

2. The method of claim 1, wherein the patient information comprises the first patient's demographic information and the first patient's calendar.

3. The method of claim 1, wherein the first calendar alias comprises a unique identifier of the first patient.

4. The method of claim 1, wherein the first calendar alias is associated with the first patient's email address.

5. The method of claim 1, further comprising: validating the first meeting request by the patient information management system.

6. The method of claim 1, further comprising: alerting the first patient about the first meeting request.
